(19) United States
(12) Patent Application Publication
(10) Pub. No.: US 2009/0220465 A1
(43) Pub. Date: Sep. 3, 2009

(54) METHODS AND COMPOSITIONS FOR MODULATION OF STEM CELL AGING

(75) Inventors: David T. Scadden, Weston, MA (US); Viktor Janzen, Bonn (DE); Randolf Forkert, Bonn (DE); Norman E. Sharpless, Chapel Hill, NC (US)

Correspondence Address: EWARDS ANGELL PALMER & DODGE LLP, P.O. BOX 55874, BOSTON, MA, 02205, US

(73) Assignees: THE GENERAL HOSPITAL CORPORATION, Boston, MA (US); UNIVERSITY OF NORTH CAROLINA AT CHAPEL HILL, Chapel Hill, NC (US)

(21) Appl. No.: 12/092,794

(22) PCT Filed: Nov. 7, 2006
(86) PCT No.: PCT/US06/43430

§ 371 Date: Mar. 2, 2009

Related U.S. Application Data
(60) Provisional application No. 60/734,336, filed on Nov. 7, 2005.

(51) Int. Cl. A61K 35/12; C12N 5/00; A61K 38/43

(52) U.S. Cl. 424/93.7; 435/375; 424/94.1

(57) ABSTRACT

Methods are described for promoting or maintaining self-renewal of a stem cell expressing or expected to express p16^{INK4a} employing p16^{INK4a} inhibitors. Methods are also described for increasing the amount of self-renewing stem cells in a non-infant subject, as well as for enhancing engraftment of a stem cell expressing p16^{INK4a}. Additionally, methods are described for identifying p16^{INK4a} inhibitors.

### RELATED APPLICATIONS/PATENTS & INCORPORATION BY REFERENCE

This application claims priority to U.S. Provisional Application Ser. No. 60/734,336, filed Nov. 7, 2005, the contents of which are incorporated herein by reference.

### GOVERNMENT SUPPORT

The work leading to the present invention was funded in part by grant numbers 5R01 HL65909 and 5 R01 DK50234, from the United States National Institutes of Health. Accordingly, the United States Government may have certain rights to this invention.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

### BACKGROUND OF THE INVENTION

The mammalian INK4a/ARF locus (cdkn2a) encodes two linked tumor suppressor proteins, the cyclin dependent kinase inhibitor p16^{INK4a} and ARF, a potent regulator of p53 stability. The two open reading frames encoding p16^{INK4a} and ARF have different promoters and first exons which splice into alternative reading frames in the shared exon 2, thereby generating these two cytogenetically linked, but functionally unrelated cancer-relevant proteins (Sharpless, Exp. Gerontol. 39, 1751-1759 (2004)). Deletion of the INK4a/ARF locus is observed with high frequency in a variety of malignancies (Rocco, J. W. et al., Exp. Cell Res. 264, 42-55 (2001)). In multiple tissues of young humans and rodents, 16^{INK4a} is virtually not detectable, while its expression dramatically increases with age (Krishnamurthy, J. et al. J. Clin. Invest. 114, 1299-1307 (2004)) (Zindy, F., et al., Oncogene 15, 203-211 (1997)). Elevated p16^{INK4a} expression has been observed in cells with replicative senescence induced by a variety of stimuli (e.g. oxidative stress, oncogene activation and telomere shortening) (Campisi, J. Cellular, Trends Cell Biol. 11, S27-31 (2001)). In addition, many human cell types acquire high levels of p16^{INK4a} expression during culture conditions that promote replicative senescence, and senescence is delayed or abrogated in many cultured cell types by p16^{INK4a} inactivation (Campisi, J. Cellular, Trends Cell Biol. 11, S27-31 (2001)). Increasing evidence suggests senescence increases with aging and induces a decline in stem cell function, including stem cell self-renewal (Ogden, D. A. et al. Transplantation 22, 287-293 (1976)) (Morrison, S. J., Wandycz, et al. Nat. Med. 2, 1011-1016 (1996) (Liang, Y., Van Zant, G. et al. Blood (2005)).

Although p16^{INK4a} expression has recently been defined as a molecular accompaniment of aging in multiple tissues, the role of p16^{INK4a} in governing the age-dependent decline in stem cell function was heretofore unknown.

### SUMMARY OF THE INVENTION

It has now been determined that p16^{INK4a} is expressed in a primitive, quiescent fraction of non-infant stem cells (e.g., hematopoietic stem cells). Deficiencies in p16^{INK4a} improve stem cell self-renewal in an age-related manner without perturbing stem cell cycling or apoptosis. It has further been determined that p16^{INK4a} deficient hematopoietic stem cells from non-infant subjects are able to provide hematopoietic reconstitution and improved survival following bone marrow transplantation. Thus, it is now understood that p16^{INK4a} participates in the stem cell aging phenotype and that inhibition of p16^{INK4a} can ameliorate the physiologic impact of aging on stem cells.

In one aspect, the invention provides a method of promoting self-renewal of a stem cell that expresses p16^{INK4a}, the method comprising the steps of contacting the stem cell with an effective amount of an inhibitor of p16^{INK4a}, thereby promoting self-renewal of the stem cell.

In another aspect, the invention provides a preventative method of maintaining self-renewal of a stem cell that does not express p16^{INK4a}, the method comprising contacting the stem cell with an inhibitor of p16^{INK4a}, thereby maintaining self-renewal of the stem cell. The stem cell can be contacted with the inhibitor of p16^{INK4a} ex vivo or in vivo. Preferably, the stem cell is that of a non-infant subject.

In yet another aspect, the invention provides a method for enhancing engraftment of a stem cell that expresses p16^{INK4a} into a tissue of a subject, the method comprising: contacting the stem cell with an effective amount of an inhibitor of p16^{INK4a} ex vivo; and providing the stem cell to the subject, thereby enhancing engraftment of the stem cell into a tissue of a subject. The tissue preferably comprises bone marrow.

In one embodiment of the invention, the inhibitor of p16^{INK4a} reduces the expression of p16^{INK4a}. The inhibitor of p16^{INK4a} that reduces the expression of p16^{INK4a} includes but is not limited to a compound that can destabilize or reduce the levels of p16^{INK4a} mRNA, a compound that can reduce translation of p16^{INK4a} mRNA, a compound that can hypermethylate p16^{INK4a}, telomerase reverse transcriptase (hTERT), an inhibitor of DNA binding/differentiation (Id, or Id-1), latent membrane protein (LMP1), helix-loop-helix transcription factor TAL1/SCL, dioxin, and cyclo-oxygenase 2 (COX-2).

In another embodiment of the invention, the inhibitor of p16^{INK4a} reduces the activity of p16^{INK4a}. The inhibitor of p16^{INK4a} that reduces the activity of p16^{INK4a} includes but is not limited to a p16^{INK4a} antibody, a compound that can hypermethylate p16^{INK4a}, telomerase reverse transcriptase (hTERT), cutaneous human papillomavirus type 16 (HPV16) E7 protein, and cyclin D1.

In yet another embodiment of the invention, the stem cell is a bone marrow derived stem cell or a hematopoietic stem cell.

In yet another embodiment of the invention, the stem cell is a mesenchymal, skin, neural, intestinal, liver, cardiac, prostate, mammary, kidney, pancreatic, retinal or lung stem cell.

In yet another embodiment of the invention, the expression of hes-1 and gfi-1 can be increased in the stem cell contacted with the inhibitor of p16^{INK4a}.

In yet another aspect, the invention provides a method of increasing the amount of self-renewing stem cells in a non-infant subject in need thereof, the method comprising the steps of: contacting an isolated population of cells comprising stem cells with an effective amount of an inhibitor of p16^{INK4a} ex-vivo; and administering the cells to the non-infant subject, thereby increasing the amount of self-renewing stem cells in the non-infant subject.

In one embodiment of the invention, the population of cells is obtained from the non-infant subject. In yet another embodiment of the invention, the population of cells comprises bone marrow cells. The population of cells can be Lin⁻, cKit⁻ and Sca1⁺. The expression of hes-1 and gfi-1 can be increased in the stem cells contacted with the inhibitor of p16^{INK4a}.

In another embodiment of the invention, the non-infant subject is a human.

In yet another embodiment of the invention, the non-infant subject is at least 18 years old.

In yet another embodiment of the invention, the stem cells are administered to the non-infant subject during a bone marrow transplant.

In yet another embodiment, the subject has a disorder including but not limited to thrombocytopenia, anemia, lymphocytopenia, lymphorrhea, lymphostasis, erythrocytopenia, erythrodegenerative disorder, erythroblastopenia, leukoerythroblastosis; erythroclasis, thalassemia, myelofibrosis, thrombocytopenia, disseminated intravascular coagulation (DIC), immune thrombocytopenic purpura (ITP), HIV inducted ITP, myelodysplasia, thrombocytotic disease, thrombocytosis, neutropaenia, myelo-dysplastic syndrome, infection, mmunodeficiency, rheumatoid arthritis, lupus, immunosuppression, systemic lupus erythematosus, rheumatoid arthritis, auto-immune thyroiditis, scleroderma, or inflammatory bowel disease.

In yet another embodiment, the various treatment methods of the invention further comprise obtaining the inhibitor of p16^{INK4a}.

In yet another aspect, the invention provides a method of identifying an inhibitor of p16^{INK4a}, wherein the inhibitor promotes the self-renewal of stem cells, the method comprising: contacting a contacting an isolated population of cells comprising stem cells that express p16^{INK4a} with an agent suspected of being an inhibitor of p16^{INK4a} ; and detecting an increase in the total number of long term repopulating cells, thereby identifying an inhibitor of p16^{INK4a} that promotes the self-renewal of the stem cells. In yet another aspect, the invention further comprises obtaining the agent suspected of being an inhibitor of p16^{INK4a}.

In one embodiment of the invention, the population of cells is obtained from a non-infant subject. In another embodiment of the invention, the population of cells comprises bone marrow cells. The population of cells can be Lin⁻, cKit⁻ and Sca1⁺. The expression of hes-1 and gfi-1 can be increased in the stem cells contacted with p16^{INK4a}.

In yet another aspect, the invention provides kits or packaged pharmaceuticals for use in practicing the methods of the invention.

In one embodiment, the invention provides a kit or packaged pharmaceutical for promoting self-renewal of a stem cell that expresses p16^{INK4a} comprising an inhibitor of p16^{INK4a}, and instructions for using the inhibitor of p16^{INK4a} to promote self-renewal of the stem cell that expresses p16^{INK4a} in accordance with the methods of the invention.

In another embodiment, the invention provides a kit or packaged pharmaceutical for increasing the amount of self-renewing stem cells in a non-infant subject in need thereof comprising an inhibitor of p16^{INK4a}, and instructions for using the inhibitor of p16^{INK4a} to increase the amount of self-renewing stem cells in the non-infant subject in need thereof in accordance with the methods of the invention.

In yet another embodiment, the invention provides a kit or packaged pharmaceutical for maintaining self-renewal of a stem cell that does not express p16^{INK4a} comprising an inhibitor of p16^{INK4a}, and instructions for using the inhibitor of p16^{INK4a} to maintain self-renewal of the stem cell that does not express p16^{INK4a}, in accordance with the methods of the invention.

In yet another embodiment, the invention provides a kit or packaged pharmaceutical for enhancing engraftment of a stem cell that expresses p16^{INK4a} into a tissue of a subject comprising an inhibitor of p16^{INK4a}, and instructions for using the inhibitor of p16^{INK4a} to enhance engraftment of a stem cell that expresses p16^{INK4a} into a tissue of the subject in accordance with the methods of the invention.

Other aspects of the invention are described in the following disclosure, and are within the ambit of the invention.

### BRIEF DESCRIPTION OF THE FIGURES

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1a shows immunoblots depicting gene expression analysis of p16^{INK4a} and ARF in sorted subpopulations of primitive hematopoietic cells of young and old FVB/n mice.

FIG. 1b shows FACS plots depicting Sca1 and c-Kit staining gated on lineage negative cells. Percentages indicate the frequency in whole bone marrow of one representative experiment (young FVB/n mice=8 weeks, old FVB/n mice=63 weeks).

FIG. 1c shows, in bar graph form, the results of an analysis of changes in CFC-frequency with aging.

FIG. 1d shows graphs depicting the results of competitive repopulation assay following the change in number of long term repopulating hematopoietic stem cells compared with wild type control. Frequency was determined using Poisson distribution (old KO vs. WT p21 0.04).

FIG. 1e shows, in bar graph form, quantitation of the rate of proliferation in primitive hematopoietic subpopulations, as affected by the presence or absence of p16^{INK4a}.

FIG. 2a shows two graphs depicting the age-dependent effect of p16^{INK4a} on stem cell self-renewal potential in terms of their survival over time relative to their wild type counterpart.

FIG. 2b shows a series of bar graphs depicting a quantification of peripheral blood leukocytes and thrombocytes over transplantation cycles.

FIG. 3a shows a series of bar graphs depicting the age-dependent effect of p16^{INK4a} on expression of self-renewal-associated genes in primitive subpopulations of bone marrow cells (Lin−c-Kit−Sca1+ and Lin−c-Kit+Sca1+).

FIG. 3b provides a schematic depiction of the coding sequence of the human papillomavirus transforming protein HPV16-E7 subcloned into the retroviral plasmid MSCV, as well as of an empty MSCV plasmid (MSCV-GFP) and a mutant variant of HPV-E7 with an inability to bind to Rb-protein MSCV-e7(Δ21-24). The bar graph below the depicted constructs shows the relative expression of hes-1, bmi-1, and gfi-1 for the three constructs. Data are presented as changes of relative expression normalized to hprt-1.

FIG. 3c schematically depicts a proposed model for the role of p16^{INK4a} in regulation of hematopoietic stem cell self-renewal. p16^{INK4a} binds to cdk4/cdk6 and inhibits the kinase activity of Cyclin D and with consecutive accumulation of hypophosphorylated Rb that binds transcription factors of the E2F family and suppresses the transcriptional activity of downstream genes. The effect of E7-expression led to a by-pass of the p16^{INK4a} effect on Rb phosphorylation and revealed Rb-mediated suppression of hes-1 expression by p16^{INK4a}. Suppression of gfi-1 expression by p16^{INK4a} might be due to a non Rb-mediated pathway.

FIGS. 4A and 4B show a series of bar graphs depicting the analysis of peripheral blood counts and bone marrow mononuclear cells in young and old WT and p16^{INK4a -/-} mice.

FIG. 5A depicts the change in survival assayed in recipient mice of whole bone marrow cell transplantation over time (n=10, p=n.s.). FIG. 5C depicts, in bar graph form, the change in production of CFC in the same mice after the 3^{rd} cycle of 5-FU administration (n=3, p=n.s.).

FIG. 6 shows, in bar graph form, staining of freshly isolated bone marrow for lineage negative, Sca-1 positive, c-Kit positive cells, as well as co-staining with Annexin V and DAPI. Apoptotic cells were defined as the Annexin V positive and DAPI negative fraction of LKS cells (n=5, p=n.s.).

### DETAILED DESCRIPTION OF THE INVENTION

### I. Definitions

The term “allogeneic,” as used herein, refers to cells of the same species that differ genetically to the cell in comparison.

The term “autologous,” as used herein, refers to cells from the same subject.

The term “engraft” as used herein refers to the process of stem cell incorporation into a tissue of interest in vivo through contact with existing cells of the tissue.

The term “non-infant subject” as used herein refers to a subject that is no longer required to nurse. Where the non-infant subject is a human, he or she is at least 6 months of age.

The term “obtaining” as in “obtaining the p16^{INK4a} inhibitor” as used herein is intended to include purchasing, synthesizing or otherwise acquiring the diagnostic agent (or indicated substance or material).

The term “p16^{INK4a} inhibitor” as used herein refers to an agent that reduces, either by decreasing or by eliminating entirely, the expression or activity of p16^{INK4a}.

The term “self-renewal” as used herein refers to the process by which a stem cell divides to generate one (asymmetric division) or two (symmetric division) daughter cells with development potentials that are indistinguishable from those of the mother cell. Self-renewal involves both proliferation and the maintenance of an undifferentiated state.

The term “stem cells” as used herein refers to multipotent or pluripotent cells having the capacity to self-renew and to differentiate into multiple cell lineages.

The term “subject” as used herein refers to any member of the class mammalia, including humans, domestic and farm animals, and zoo, sports or pet animals, such as mouse, rabbit, pig, sheep, goat, cattle and higher primates.

The term “syngeneic,” as used herein, refers to cells of a different subject that are genetically identical to the cell in comparison.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

The term “xenogenic,” as used herein, refers to cells of a different species to the cell in comparison.

In this disclosure, the terms “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### II. Compositions and Methods of the Invention

Uses of p16^{INK4a} Inhibitors

Stem cells may, according to the invention, be contacted ex vivo with a p16^{INK4a} inhibitor to promote stem cell renewal. Once treated with a p16 INK4a inhibitor according to the methods of the invention, as described herein, stem cells can be returned to the body to supplement, replenish, etc. a patient's stem cell population. Such p16^{INK4a} treatment of the stem cells will increase the stem cell pool and enhance stem cell engraftment potential upon administration.

Preferably, isolated cells are treated with the p16^{INK4a} inhibitor prior to the initiation of a therapeutic regimen likely to cause stress to the cells (for example, prior to expansion and re-implantation or transplantation), as it is believed that p16^{INK4a}, if not already expressed, can be induced as a result of stress. In this regard, it is also desirable to treat cells that do not yet express p16^{INK4a}, as such treatment can guard against the induction of undesired p16^{INK4a} expression.

In some embodiments, an effective amount of the p16^{INK4a} inhibitor can be directly administered to subjects in vivo. Under such conditions, the inhibitor works in vivo to preserve and ultimately increase the stem cell pool. Suitable inhibitors can be administered by a variety of routes. Methods of administration, generally speaking, may be practiced using any mode of administration that is medically acceptable, meaning any mode that produces effective levels of the active compounds without causing clinically unacceptable adverse effects. Such modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes. The term “parenteral” includes subcutaneous, intravenous, intramuscular, intraperitoneal, or infusion.

p16^{INK4a} inhibitors that can be used in accordance with methods of the invention include all such agents known in the art to reduce the expression or activity of p16^{INK4a}. Such agents include, without limitation, p16^{INK4a} antibodies, any compound leading to the hypermethylation of p16^{INK4a} (Zochbauer-Muller, S., et al. 2001 Cancer Res 61(1):249-55; Wong, L., et al. 2002 Lung Cancer 38(2): 131-6), telomerase reverse transcriptase (hTERT) (Veitomnaki, N., et al. 2003 FASEB J 17(6):764-6; Taylor, L. M., et al., 2004 J Biol Chem 279(42):43634-45), cutaneous human papillomavirus type 16 (HPV16) E7 protein (Giarre, M., et al. 2001 J Virol 75(10):4705-12), inhibitor of DNA binding/differentiation (Id, or Id-1) (Sakurai, D., et al. 2004 J Immunol 173(9):5801-9; Lee, T. K., et al. 2003 Carcinogenesis 24(11): 1729-36), latent membrane protein (LMP1) (Yang, X., et al. 2000 Oncogene 19(16):2002-13), helix-loop-helix transcription factor TAL1/SCL (Hansson, A., et al. 2003 Biochem Biophys Res Commun 312(4):1073-81), cyclin D1 (D'Amico, M., et al. 2004 Cancer Res 64(12):4122-30), dioxin (Ray, S. S., et al. 2004 J Biol Chem 279(26):27187-93), and cyclo-oxygenase 2 (COX-2) (Crawford, Y. G., et al. 2004 Cancer Cell 5(3):263-73).

p16^{INK4a} inhibitors that can be used in accordance with methods of the invention to reduce the expression of p16^{INK4a} include compounds that can destabilize or reduce the levels of p16^{INK4a} mRNA. For example, RNAi-mediated gene silencing by shRNA, siRNA, or microRNA that target p16^{INK4a} mRNA can be used to destabilize p16^{INK4a} mRNA. RNAi-mediated gene silencing is initiated by introducing into cells either synthetic small interfering RNA (siRNA) or longer double-stranded RNA molecules which are secondarily processed into siRNA or microRNA (miRNA) that target a specific mRNA sequence (e.g., p16^{INK4a} mRNA). Small stem-loop RNAs yield short-hairpin RNAs (shRNA) can also be introduced into cells and further processed to target a specific mRNA sequence. ShRNAs are processed by the same mechanism as endogenous miRNA precursors and exported to the cytoplasm by the karyopherin exportin-5, where 21 to 28-nucleotide (nt) duplex fragments with 3′ di-nucleotide overhangs are then generated by the RNase III-like enzyme Dicer. Upon unwinding within the RNA-induced silencing complex and annealing to the target sequence, the latter is cleaved by the slicer Argonaut-2 protein and further digested by cytoplasmic exonuclease. Precursor miRNAs are also processed by Dicer but incorporated in miRNPs that target a specific mRNA sequence to inhibit its translation.

p16^{INK4a} inhibitors that can be used in accordance with methods of the invention to reduce p16^{INK4a} expression also include compounds that can reduce translation of p16^{INK4a}. For example, complementary strands of RNA (antisense RNA) that anneal to p16^{INK4a} mRNA can be introduced into cells to block translation of p16^{INK4a} mRNA.

The p16^{INK4a} inhibitor may be supplied along with additional reagents in a kit. The kits can include instructions for the treatment regime or assay, reagents, equipment (test tubes, reaction vessels, needles, syringes, etc.) and standards for calibrating or conducting the treatment or assay. The instructions provided in a kit according to the invention may be directed to suitable operational parameters in the form of a label or a separate insert. Optionally, the kit may further comprise a standard or control information so that the test sample can be compared with the control information standard to determine whether a consistent result is achieved.

### Stem Cells

Stem cells of the present invention include all those known in the art that have been identified in mammalian organs or tissues. The best characterized is the hematopoietic stem cell. The hematopoietic stem cell, isolated from bone marrow, blood, cord blood, fetal liver and yolk sac, is the progenitor cell that generates blood cells or following transplantation reinitiates multiple hematopoietic lineages and can reinitiate hematopoiesis for the life of a recipient. (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827; Hill, B., et al. 1996.) When transplanted into lethally irradiated animals or humans, hematopoietic stem cells can repopulate the erythroid, neutrophil-macrophage, megakaryocyte and lymphoid hematopoietic cell pool. In vitro, hematopoietic stem cells can be induced to undergo at least some self-renewing cell divisions and can be induced to differentiate to the same lineages observed in vivo.

It is well known in the art that hematopoietic cells include pluripotent stem cells, multipotent progenitor cells (e.g., a lymphoid stem cell), and/or progenitor cells committed to specific hematopoietic lineages. The progenitor cells committed to specific hematopoietic lineages may be of T cell lineage, B cell lineage, dendritic cell lineage, Langerhans cell lineage and/or lymphoid tissue-specific macrophage cell lineage.

Hematopoietic stem cells can be obtained from blood products. A “blood product” as used in the present invention defines a product obtained from the body or an organ of the body containing cells of hematopoietic origin. Such sources include unfractionated bone marrow, umbilical cord, peripheral blood, liver, thymus, lymph and spleen. It will be apparent to those of ordinary skill in the art that all of the aforementioned crude or unfractionated blood products can be enriched for cells having “hematopoietic stem cell” characteristics in a number of ways. For example, the blood product can be depleted from the more differentiated progeny. The more mature, differentiated cells can be selected against, via cell surface molecules they express. Additionally, the blood product can be fractionated selecting for CD34⁺ cells. CD34⁺ cells are thought in the art to include a subpopulation of cells capable of self-renewal and pluripotentiality. Such selection can be accomplished using, for example, commercially available magnetic anti-CD34 beads (Dynal, Lake Success, N.Y.). Unfractionated blood products can be obtained directly from a donor or retrieved from cryopreservative storage.

In preferred embodiments of the invention, the hematopoietic stem cells may be harvested prior to treatment with p16^{INK4a} inhibitors. “Harvesting” hematopoietic progenitor cells is defined as the dislodging or separation of cells from the matrix. This can be accomplished using a number of methods, such as enzymatic, non-enzymatic, centrifugal, electrical, or size-based methods, or preferably, by flushing the cells using media (e.g. media in which the cells are incubated). The cells can be further collected, separated, and further expanded generating even larger populations of differentiated progeny.

Methods for isolation of hematopoietic stem cells are well-known in the art, and typically involve subsequent purification techniques based on cell surface markers and functional characteristics. The hematopoietic stem and progenitor cells can be isolated from bone marrow, blood, cord blood, fetal liver and yolk sac, and give rise to multiple hematopoietic lineages and can reinitiate hematopoiesis for the life of a recipient. (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827; Hill, B., et al. 1996.) For example, for isolating hematopoietic stem and progenitor cells from peripheral blood, blood in PBS is loaded into a tube of Ficoll (Ficoll-Paque, Amersham) and centrifuged at 1500 rpm for 25-30 minutes. After centrifugation the white center ring is collected as containing hematopoietic stem cells.

Stem cells of the present invention also include embryonic stem cells. The embryonic stem (ES) cell has unlimited self-renewal and pluripotent differentiation potential (Thomson, J. et al. 1995; Thomson, J. A. et al. 1998; Shamblott, M. et al. 1998; Williams, R. L. et al. 1988; Orkin, S. 1998; Reubinoff, B. E., et al. 2000). These cells are derived from the inner cell mass (ICM) of the pre-implantation blastocyst (Thomson, J. et al. 1995; Thomson, J. A. et al. 1998; Martin, G. R. 1981), or can be derived from the primordial germ cells from a post-implantation embryo (embryonal germ cells or EG cells). ES and/or EG cells have been derived from multiple species, including mouse, rat, rabbit, sheep, goat, pig and more recently from human and human and non-human primates (U.S. Pat. Nos. 5,843,780 and 6,200,806).

Embryonic stem cells are well known in the art. For example, U.S. Pat. Nos. 6,200,806 and 5,843,780 refer to primate, including human, embryonic stem cells. U.S. Patent Applications Nos. 20010024825 and 20030008392 describe human embryonic stem cells. U.S. Patent Application No. 20030073234 describes a clonal human embryonic stem cell line. U.S. Pat. No. 6,090,625 and U.S. Patent Application No. 20030166272 describe an undifferentiated cell that is stated to be pluripotent. U.S. Patent Application No. 20020081724 describes what are stated to be embryonic stem cell derived cell cultures.

Stem cells of the present invention also include mesenchymal stem cells. Mesenchymal stem cells, or “MSCs” are well known in the art. MSCs, originally derived from the embryonal mesoderm and isolated from adult bone marrow, can differentiate to form muscle, bone, cartilage, fat, marrow stroma, and tendon. During embryogenesis, the mesoderm develops into limb-bud mesoderm, tissue that generates bone, cartilage, fat, skeletal muscle and endothelium. Mesoderm also differentiates to visceral mesoderm, which can give rise to cardiac muscle, smooth muscle, or blood islands consisting of endothelium and hematopoietic progenitor cells. Primitive mesodermal or MSCs, therefore, could provide a source for a number of cell and tissue types. A number of MSCs have been isolated. (See, for example, Caplan, A., et al., U.S. Pat. No. 5,486,359; Young, H., et al., U.S. Pat. No. 5,827,735; Caplan, A., et al., U.S. Pat. No. 5,811,094; Bruder, S., et al., U.S. Pat. No. 5,736,396; Caplan, A., et al., U.S. Pat. No. 5,837,539; Masinovsky, B., U.S. Pat. No. 5,837,670; Pittenger, M., U.S. Pat. No. 5,827,740; Jaiswal, N., et al., (1997). J. Cell Biochem. 64(2):295-312; Cassiede P., et al., (1996). J Bone Miner Res. 9:1264-73; Johnstone, B., et al., (1998) Exp. Cell Res. 1:265-72; Yoo, et al.,(1998) Bon Joint Surg Am. 12:1745-57; Gronthos, S., et al., (1994). Blood 84:4164-73); Pittenger, et al., (1999). Science 284:143-147.

Mesenchymal stem cells are believed to migrate out of the bone marrow, to associate with specific tissues, where they will eventually differentiate into multiple lineages. Enhancing the growth and maintenance of mesenchymal stem cells, in vitro or ex vivo will provide expanded populations that can be used to generate new tissue, including breast, skin, muscle, endothelium, bone, respiratory, urogenital, gastrointestinal connective or fibroblastic tissues.

Stem cells of the present invention also include all adult stem cells known in the art, such as skin, neural, intestinal, liver, cardiac, prostate, mammary, kidney, pancreatic, retinal or lung stem cells.

Stem cells used according to methods of the invention can be treated with p16^{INK4a} as either purified or non-purified fractions prior to administration. Biological samples may comprise mixed populations of cells, which can be purified to a degree sufficient to produce a desired effect. Those skilled in the art can readily determine the percentage of stem cells or their progenitors in a population using various well-known methods, such as fluorescence activated cell sorting (FACS). Purity of the stem cells can be determined according to the genetic marker profile within a population. Dosages can be readily adjusted by those skilled in the art (e.g., a decrease in purity may require an increase in dosage).

In several embodiments, it will be desirable to first purify the cells. Stem cells of the invention preferably comprise a population of cells that have about 50-55%, 55-60%, 60-65% and 65-70% purity (e.g., non-stem and/or non-progenitor cells have been removed or are otherwise absent from the population). More preferably the purity is about 70-75%, 75-80%, 80-85%; and ever more preferably the purity is about 85-90%, 90-95%, and 95-100%. Purified populations of stem cells of the invention can be contacted with p16^{INK4a} inhibitor before, after or concurrently with purification steps and administered to the subject.

Once obtained from the desired source, contacting of the cells with the p16^{INK4a} inhibitor will typically occur in the culture. Employing the culture conditions described in greater detail below, it is possible to preserve stem cells of the invention and to stimulate the expansion of stem cell number and/or colony forming unit potential. In all of the in vitro and ex vivo culturing methods according to the invention, except as otherwise provided, the media used is that which is conventional for culturing cells. Appropriate culture media can be a chemically defined serum-free media such as the chemically defined media RPMI, DMEM, Iscove's, etc or so-called “complete media”. Typically, serum-free media are supplemented with human or animal plasma or serum. Such plasma or serum can contain small amounts of hematopoietic growth factors. The media used according to the present invention, however, can depart from that used conventionally in the prior art. Suitable chemically defined serum-free media are described in U.S. Ser. No. 08/464,599 and WO96/39487, and “complete media” are described in U.S. Pat. No. 5,486,359.

Treatment of the stem cells of the invention with p16^{INK4a} inhibitors may involve variable parameters depending on the particular type of inhibitor used. For example, ex vivo treatment of stem cells with RNAi constructs may have a rapid effect (e.g., within 1-5 hours post transfection) while treatment with a chemical agent may require extended incubation periods (e.g., 24-48 hours). It is also possible to co-culture the stem cells treated according to the invention with additional agents that promote stem cell maintenance and expansion. It is well within the level of ordinary skill in the art for practitioners to vary the parameters accordingly.

The growth agents of particular interest in connection with the present invention are hematopoietic growth factors. By hematopoietic growth factors, it is meant factors that influence the survival or proliferation of hematopoietic stem cells. Growth agents that affect only survival and proliferation, but are not believed to promote differentiation, include the interleukins 3, 6 and 11, stem cell factor and FLT-3 ligand. The foregoing factors are well known to those of ordinary skill in the art and most are commercially available. They can be obtained by purification, by recombinant methodologies or can be derived or synthesized synthetically.

Thus, when cells are cultured without any of the foregoing agents, it is meant herein that the cells are cultured without the addition of such agent except as may be present in serum, ordinary nutritive media or within the blood product isolate, unfractionated or fractionated, which contains the hematopoietic stem and progenitor cells.

Isolated stem cells of the invention can be genetically altered. For example, the stem cells described herein can be genetically modified to knock out p16^{INK4a}, resulting in p16^{INK4a-/-} cells. Alternatively, stem cells of the invention can be engineered to express a gene encoding a protein or mRNA (e.g., siRNA) that suppresses expression of a p16^{INK4a}.

Genetic alteration of a stem cell includes all transient and stable changes of the cellular genetic material, which are created by the addition of exogenous genetic material. Examples of genetic alterations include any gene therapy procedure, such as introduction of a functional gene to replace a mutated or non-expressed gene, introduction of a vector that encodes a dominant negative gene product, introduction of a vector engineered to express a ribozyme and introduction of a gene that encodes a therapeutic gene product. Exogenous genetic material includes nucleic acids or oligonucleotides, either natural or synthetic, that are introduced into the stem cells. The exogenous genetic material may be a copy of that which is naturally present in the cells, or it may not be naturally found in the cells. It typically is at least a portion of a naturally occurring gene which has been placed under operable control of a promoter in a vector construct.

Various techniques may be employed for introducing nucleic acids into cells. Such techniques include transfection of nucleic acid-CaPO₄ precipitates, transfection of nucleic acids associated with DEAE, transfection with a retrovirus including the nucleic acid of interest, liposome mediated transfection, and the like. For certain uses, it is preferred to target the nucleic acid to particular cells. In such instances, a vehicle used for delivering a nucleic acid according to the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. For example, where liposomes are employed to deliver the nucleic acids of the invention, proteins which bind to a surface membrane protein associated with endocytosis may be incorporated into the liposome formulation for targeting and/or to facilitate uptake. Such proteins include proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

One method of introducing exogenous genetic material into cells involves transducing the cells in situ on the matrix using replication-deficient retroviruses. Replication-deficient retroviruses are capable of directing synthesis of all virion proteins, but are incapable of making infectious particles. Accordingly, these genetically altered retroviral vectors have general utility for high-efficiency transduction of genes in cultured cells, and specific utility for use in the method of the present invention. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in the art.

Because viruses insert efficiently a single copy of the gene encoding the therapeutic agent into the host cell genome, retroviruses permit the exogenous genetic material to be passed on to the progeny of the cell when it divides. In addition, gene promoter sequences in the LTR region have been reported to enhance expression of an inserted coding sequence in a variety of cell types. However, using a retrovirus expression vector may result in (1) insertional mutagenesis, i.e., the insertion of the therapeutic gene into an undesirable position in the target cell genome which, for example, leads to unregulated cell growth and (2) the need for target cell proliferation in order for the therapeutic gene carried by the vector to be integrated into the target genome. Despite these apparent limitations, delivery of a therapeutically effective amount of a therapeutic agent via a retrovirus can be efficacious if the efficiency of transduction is high and/or the number of target cells available for transduction is high.

Yet another viral candidate useful as an expression vector for transformation of cells is the adenovirus, a double-stranded DNA virus. Like the retrovirus, the adenovirus genome is adaptable for use as an expression vector for gene transduction, i.e., by removing the genetic information that controls production of the virus itself. Because the adenovirus functions usually in an extrachromosomal fashion, the recombinant adenovirus does not have the theoretical problem of insertional mutagenesis. On the other hand, adenoviral transformation of a target cell may not result in stable transduction. However, more recently it has been reported that certain adenoviral sequences confer intrachromosomal integration specificity to carrier sequences, and thus result in a stable transduction of the exogenous genetic material.

Thus, as will be apparent to one of ordinary skill in the art, a variety of suitable vectors are available for transferring exogenous genetic material into cells. The selection of an appropriate vector to deliver an agent and the optimization of the conditions for insertion of the selected expression vector into the cell, are within the scope of one of ordinary skill in the art without the need for undue experimentation. The promoter characteristically has a specific nucleotide sequence that is desirable to initiate transcription. Optionally, the exogenous genetic material further includes additional sequences (i.e., enhancers) employed to obtain the desired gene transcription activity. For the purpose of this discussion an “enhancer” is simply any non-translated DNA sequence which works contiguous with the coding sequence (in cis) to change the basal transcription level dictated by the promoter. Preferably, the exogenous genetic material is introduced into the cell genome immediately downstream from the promoter so that the promoter and coding sequence are operatively linked so as to permit transcription of the coding sequence. A preferred retroviral expression vector includes an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., 1991, Proc. Natl. Acad. Sci. USA, 88:4626-4630), adenosine deaminase, phosphoglycerol kinase (PGK), pyruvate kinase, phosphoglycerol mutase, the actin promoter (Lai et al., 1989, Proc. Natl. Acad. Sci. USA, 86:10006-10010), and other constitutive promoters known to those of skill in the art. In addition, many viral promoters function constitutively in eukaryotic cells. These include: the early and late promoters of SV40; the long terminal repeats (LTRS) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionine promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of an agent in the genetically modified cell. Selection and optimization of these factors for delivery is deemed to be within the scope of one of ordinary skill in the art without undue experimentation, taking into account the above-disclosed factors.

In addition to at least one promoter and at least one heterologous nucleic acid, the expression vector preferably includes a selection gene, for example, a neomycin resistance gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene and/or signal sequence is deemed to be within the scope of one of ordinary skill in the art without undue experimentation.

### Treatment Methods

The methods of the invention can be used to treat any disease or disorder in which it is desirable to increase the amount of stem cells and support the maintenance or survival of stem cells. Preferably, the stem cells are hematopoietic stem cells of a non-infant subject.

Frequently, subjects in need of the inventive treatment methods will be those undergoing or expecting to undergo an immune cell depleting treatment such as chemotherapy. Most chemotherapy agents used act by killing all cells going through cell division. Bone marrow is one of the most prolific tissues in the body and is therefore often the organ that is initially damaged by chemotherapy drugs. The result is that blood cell production is rapidly destroyed during chemotherapy treatment, and chemotherapy is terminated to allow the hematopoietic system to replenish the blood cell supplies before a patient is re-treated with chemotherapy.

Thus, methods of the invention can be used, for example, to treat patients requiring a bone marrow transplant or a hematopoietic stem cell transplant, such as cancer patients undergoing chemo and/or radiation therapy. Methods of the present invention are particularly useful in the treatment of patients undergoing chemotherapy or radiation therapy for cancer, including patients suffering from myeloma, non-Hodgkin's lymphoma, Hodgkin's lymphoma, or leukemia.

Preferably, the receiving subject and the donating subject are non-infant subjects, as the beneficial effect of p16^{INK4a} inhibition is not expected in infant subjects. Preferably, the non-infant subjects are human.

Disorders treated by methods of the invention can be the result of an undesired side effect or complication of another primary treatment, such as radiation therapy, chemotherapy, or treatment with a bone marrow suppressive drug, such as zidovadine, chloramphenical or ganciclovir. Such disorders include neutropenias, anemias, thrombocytopenia, and immune dysfunction. In addition, methods of the invention can be used to treat damage to the bone marrow caused by unintentional exposure to toxic agents or radiation.

Methods of the invention can further be used as a means to increase the amount of mature cells derived from hematopoietic stem cells (e.g., erythrocytes). For example, disorders or diseases characterized by a lack of blood cells, or a defect in blood cells, can be treated by increasing the pool of hematopoietic stem cells. Such conditions include thrombocytopenia (platelet deficiency), and anemias such as aplastic anemia, sickle cell anemia, fanconi's anemia, and acute lymphocytic anemia. In addition to the above, further conditions which can benefit from treatment using methods of the invention include, but are not limited to, lymphocytopenia, lymphorrhea, lymphostasis, erythrocytopenia, erythrodegenerative disorders, erythroblastopenia, leukoerythroblastosis; erythroclasis, thalassemia, myelofibrosis, thrombocytopenia, disseminated intravascular coagulation (DIC), immune (autoimmune) thrombocytopenic purpura (ITP), HIV inducted ITP, myelodysplasia; thrombocytotic disease, thrombocytosis, congenital neutropenias (such as Kostmann's syndrome and Schwachman-Diamond syndrome), neoplastic associated—neutropenias, childhood and adult cyclic neutropaenia; post-infective neutropaenia; myelo-dysplastic syndrome; and neutropaenia associated with chemotherapy and radiotherapy.

The disorder to be treated can also be the result of an infection (e.g., viral infection, bacterial infection or fungal infection) causing damage to stem cells.

Immunodeficiencies, such as T and/or B lymphocytes deficiencies, or other immune disorders, such as rheumatoid arthritis and lupus, can also be treated according to the methods of the invention. Such immunodeficiencies may also be the result of an infection (for example infection with HIV leading to AIDS), or exposure to radiation, chemotherapy or toxins.

Also benefiting from treatment according to methods of the invention are individuals who are healthy, but who are at risk of being affected by any of the diseases or disorders described herein (“at-risk” individuals). At-risk individuals include, but are not limited to, individuals who have a greater likelihood than the general population of becoming cytopenic or immune deficient. Individuals at risk for becoming immune deficient include, but are not limited to, individuals at risk for HIV infection due to sexual activity with HIV-infected individuals; intravenous drug users; individuals who may have been exposed to HIV-infected blood, blood products, or other HIV-contaminated body fluids; babies who are being nursed by HIV-infected mothers; individuals who were previously treated for cancer, e.g., by chemotherapy or radiotherapy, and who are being monitored for recurrence of the cancer for which they were previously treated; and individuals who have undergone bone marrow transplantation or any other organ transplantation, or patients anticipated to undergo chemotherapy or radiation therapy or be a donor of stem cells for transplantation.

A reduced level of immune function compared to a normal subject can result from a variety of disorders, diseases infections or conditions, including immunosuppressed conditions due to leukemia, renal failure; autoimmune disorders, including, but not limited to, systemic lupus erythematosus, rheumatoid arthritis, auto-immune thyroiditis, scleroderma, inflammatory bowel disease; various cancers and tumors; viral infections, including, but not limited to, human immunodeficiency virus (HIV); bacterial infections; and parasitic infections.

A reduced level of immune function compared to a normal subject can also result from an immunodeficiency disease or disorder of genetic origin, or due to aging. Examples of these are immunodeficiency diseases associated with aging and those of genetic origin, including, but not limited to, hyperimmunoglobulin M syndrome, CD40 ligand deficiency, IL-2 receptor deficiency, γ-chain deficiency, common variable immunodeficiency, Chediak-Higashi syndrome, and Wiskott-Aldrich syndrome.

A reduced level of immune function compared to a normal subject can also result from treatment with specific pharmacological agents, including, but not limited to chemotherapeutic agents to treat cancer; certain immunotherapeutic agents; radiation therapy; immunosuppressive agents used in conjunction with bone marrow transplantation; and immunosuppressive agents used in conjunction with organ transplantation.

Where the stem cells to be provided (ex vivo) to a subject in need of such treatment are hematopoietic stem cells, they are most commonly obtained from the bone marrow of the subject or a compatible donor. Bone marrow cells can be easily isolated using methods know in the art. For example, bone marrow stem cells can be isolated by bone marrow aspiration. U.S. Pat. No. 4,481,946, incorporated herein expressly by reference, describes a bone marrow aspiration method and apparatus, wherein efficient recovery of bone marrow from a donor can be achieved by inserting a pair of aspiration needles at the intended site of removal. Through connection with a pair of syringes, the pressure can be regulated to selectively remove bone marrow and sinusoidal blood through one of the aspiration needles, while positively forcing an intravenous solution through the other of the aspiration needles to replace the bone marrow removed from the site. The bone marrow and sinusoidal blood can be drawn into a chamber for mixing with another intravenous solution and thereafter forced into a collection bag. The heterogeneous cell population can be further purified by identification of cell-surface markers to obtain the bone marrow derived germline stem cell compositions for administration into the reproductive organ of interest.

U.S. Pat. No. 4,486,188 describes methods of bone marrow aspiration and an apparatus in which a series of lines are directed from a chamber section to a source of intravenous solution, an aspiration needle, a second source of intravenous solution and a suitable separating or collection source. The chamber section is capable of simultaneously applying negative pressure to the solution lines leading from the intravenous solution sources in order to prime the lines and to purge them of any air. The solution lines are then closed and a positive pressure applied to redirect the intravenous solution into the donor while negative pressure is applied to withdraw the bone marrow material into a chamber for admixture with the intravenous solution, following which a positive pressure is applied to transfer the mixture of the intravenous solution and bone marrow material into the separating or collection source.

It will be apparent to those of ordinary skill in the art that the crude or unfractionated bone marrow can be enriched for cells having desired “stem cell” characteristics. Some of the ways to enrich include, e.g., depleting the bone marrow from the more differentiated progeny. The more mature, differentiated cells can be selected against, via cell surface molecules they express. Enriched bone marrow immunophenotypic subpopulations include but are not limited to populations sorted according to their surface expression of Lin, cKit and Sca-1 (e.g., LK+S+ (Lin−cKit⁺Sca1⁺), LK−S+ (Lin−cKit⁺Sca1⁺), and LK+S− (Lin−cKit⁺Sca1⁺)).

Bone marrow can be harvested during the lifetime of the subject. However, harvest prior to illness (e.g., cancer) is desirable, and harvest prior to treatment by cytotoxic means (e.g., radiation or chemotherapy) will improve yield and is therefore also desirable.

### Administration of Stem Cells

Following ex vivo treatment with a suitable p16^{INK4a} inhibitor, stem cells of the invention will be administered according to methods known in the art. Such compositions may be administered by any conventional route, including injection or by gradual infusion over time. The administration may, depending on the composition being administered, for example, be, pulmonary, intravenous, intraperitoneal, intramuscular, intracavity, subcutaneous, or transdermal. The stem cells are administered in “effective amounts”, or the amounts that either alone or together with further doses produces the desired therapeutic response.

Administered cells of the invention can be autologous (“self”) or non-autologous (“non-self,” e.g., allogeneic, syngeneic or xenogeneic). Generally, administration of the cells can occur within a short period of time following p16^{INK4a} treatment (e.g. 1, 2, 5, 10, 24 or 48 hours after treatment) and according to the requirements of each desired treatment regimen. For example, where radiation or chemotherapy is conducted prior to administration, treatment, and transplantation of stem cells of the invention should optimally be provided within about one month of the cessation of therapy. However, transplantation at later points after treatment has ceased can be done with derivable clinical outcomes.

Following harvest and treatment with a suitable p16^{INK4a} inhibitor, stem cells may be combined with pharmaceutical excipients known in the art to enhance preservation and maintenance of the cells prior to administration. In some embodiments, stem cell compositions of the invention can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the cells utilized in practicing the present invention in the amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

A method to increase cell survival when introducing the cells into a subject in need thereof is to incorporate stem cells of interest into a biopolymer or synthetic polymer. Examples of biopolymer include, but are not limited to, cells mixed with fibronectin, fibrin, fibrinogen, thrombin, collagen, and proteoglycans. This could be constructed with or without included expansion or differentiation factors. Additionally, these could be in suspension, but residence time at sites subjected to flow would be nominal. Another alternative is a three-dimensional gel with cells entrapped within the interstices of the cell biopolymer admixture. Again, expansion or differentiation factors could be included with the cells. These could be deployed by injection via various routes described herein.

Those skilled in the art will recognize that the components of the compositions should be selected to be chemically inert and will not affect the viability or efficacy of the stem cells or their progenitors as described in the present invention. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of stem cells is the quantity of cells needed to achieve an optimal effect. Different scenarios may require optimization of the amount of cells injected into a tissue of interest. Thus, the quantity of cells to be administered will vary for the subject being treated. The precise determination of what would be considered an effective dose may be based on factors individual to each patient, including their size, age, sex, weight, and condition of the particular patient. As few as 100-1000 cells can be administered for certain desired applications among selected patients. Therefore, dosages can be readily ascertained by those skilled in the art from this disclosure and the knowledge in the art.

The skilled artisan can readily determine the amount of cells and optional additives, vehicles, and/or carrier in compositions and to be administered in methods of the invention. Of course, for any composition to be administered to an animal or human, and for any particular method of administration, it is preferred to determine therefore: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### Screening Assays

Screening methods of the invention can involve the identification of a p16^{INK4a} inhibitor that promotes the self-renewal of stem cells. Such methods will typically involve contacting a population of cells comprising stem cells that express p16^{INK4a} with a suspected inhibitor in culture and quantitating the number of long-term repopulating cells produced as a result. A quantitative in vivo assay (for the determination of the relative frequency of long-term repopulating stem cells) based on competitive repopulation combined with limiting dilution analysis has been previously described in Schneider, T. E., et al. (2003) PNAS 100(20):11412-11417. Similarly, Zhang, J., et al. (2005 Gene Therapy 12:1444-1452) describes the injection of NOD/SCID mice with siRNA-treated lentiviral-transduced human CD34+ cells, followed by the killing of the mice and harvesting of the bone marrow mononuclear cells. The cells were subsequently stained with anti-human leukocyte marker antibodies for FACS analysis allowing the detection of the markers (and, thus, quantitation of the cells of interest). Comparison to an untreated control can be concurrently assessed. Where an increase in the number of long-term repopulating cells is detected relative to the control, the suspected inhibitor is determined to have the desired activity.

In further embodiments, screening methods of the invention can involve the detection and quantitation of hes-1 and/or gfi-1 gene expression in stem cells. Where hes-1 and gfi-1 levels both increase in stem cells, increased stem cell self-renewal is expected.

In practicing the screening methods of the invention, it may be desirable to employ a purified population of stem cells. In other methods, the test agent is assayed using a biological sample rather than a purified population of stem cells. The term “biological sample” includes tissues, cells and biological fluids isolated from a subject, as well as tissues, cells and fluids present within a subject. Preferred biological samples include bone marrow and peripheral blood.

Increased amounts of long-term repopulating cells can be detected by an increase in gene expression of certain markers including but not limited to Hes-1, Bmi-1, Gfi-1, SLAM genes, CD51, GATA-2, Scl, P2y14, and CD34. These cells may also be characterized by a decreased or low expression of genes associated with differentiation.

The level of expression of genes of interest (e.g. hes-1, gfi-1) can be measured in a number of ways, including, but not limited to: measuring the MRNA encoded by the genes; measuring the amount of protein encoded by the genes; or measuring the activity of the protein encoded by the genes.

The level of MRNA corresponding to a gene of interest can be determined both by in situ and by in vitro formats. The isolated MRNA can be used in hybridization or amplification assays that include, but are not limited to, Southern or Northern analyses, polymerase chain reaction analyses and probe arrays. One diagnostic method for the detection of mRNA levels involves contacting the isolated MRNA with a nucleic acid molecule (probe) that can hybridize to the mRNA encoded by the gene being detected. The nucleic acid probe is sufficient to specifically hybridize under stringent conditions to mRNA or genomic DNA. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. In yet another format, bead-based analysis is employed, such as that described in J. Lu, et al. 2005 Nature 435:834-838, where DNA sequences complementary to individual miRNAs are attached to color-coded beads, and miRNAs amplified from target cells are then applied to the beads, stained, and identified via cell-sorting. A skilled artisan can adapt known mRNA detection methods for use in detecting the level of mRNA encoded by the genes of interest described herein.

The level of mRNA in a sample can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the gene of interest being analyzed.

The present invention is additionally described by way of the following illustrative, non-limiting Examples that provide a better understanding of the present invention and of its many advantages.

### EXAMPLES

### Example 1

### Analysis of Hematopoietic Stem Cells in p16^{INK4a} and p16^{INK4a-/-} Mice

Since p16^{INK4a} expression has recently been defined as a molecular accompaniment of aging in multiple tissues, whether p16^{INK4a} plays a prominent role in governing the age-dependent decline in stem cell function was investigated. (Krishnamurthy, J. et al. J. Clin. Invest. 114, 1299-1307 (2004)) Expression of p16^{INK4a} was examined in different subpopulations of mouse bone marrow in both young adult (8-12 week old) and old (52-78 week old) animals.

### Mice

FVB/n, C57B1/6 wild type and p16^{INK4a-/-} mice were bred in-house in a pathogen-free environment. The p16^{INK4a} KO mouse on FVB/n were generated as previously described (Harrison, D. E. Nat. New Biol. 237, 220-222 (1972)) and backcrossed to C57B1/6 for 6 generations. The Institutional Animal Care and Use Committee of the University of North Carolina and the Subcommittee on Research Animal Care of the Massachusetts General Hospital (MGH) approved all animal work according to federal and institutional policies and regulations.

### Retroviral Gene Transfer of LKS

cDNAs encoding HPV16-E7 and E7 A21 -24 sequence (Phelps, W. C., et al. J. Virol 66, 2418-2427 (1992)) were subcloned into the retroviral vector MSCV. Virus production and transduction of sorted LKS cells was performed as previously described (Stier, S., et al. Blood 99, 2369-2378 (2002)). Two days after virus transduction, LKS cells were sorted for GFP+ cells and cultured for 8 additional days in HSC medium with subsequent RNA-isolation and gene expression analysis.

### Cells and Cell Culture

Bone marrow was harvested as previously described (Cheng, T. et al. Science 287, 1804-1808 (2000)) and cultured in CFU-C and CFU-Mk assays according to the manufacturers' protocols (Stem Cell Technologies). Sorted LK+S+ cells were cultured in HSC medium: X-Vivo 15™ (Cambrex) supplemented with 10% detoxified BSA (StemCell Technologies, Inc.), 100 U/ml penicillin (BioWhittaker), 100 U/ml streptomycin (Cellgro), 2 mM L-glutamine (BioWhittaker), and 0.1 mM 2-mercaptoethanol (Sigma-Aldridge). Prior to virus transduction, LKS cells were cultured in presence of 50 ng/ml rmSCF, 50 ng/ml rmTPO, 50 ng/ml rmFlt-3L and 20 ng/ml rmIL3 (all from PeproTech). 24 hours after virus transduction, cells were cultured in fresh HSC medium in the presence of 10 ng/ml rmSCF, 10 ng/ml rmTPO.

### Flow Cytometric Analysis and Sorting of Subpopulations

Biotinylated anti-mouse antibodies to Mac-1α (CD11b), Gr-1(Ly-6G & 6C), Ter119 (Ly-76), CD3ε, CD4, CD8a (Ly-2), and B220 (CD45R) (BD Biosciences) were used for lineage staining. For detection and sorting, streptavidin conjugated with PE/Cy7 (BD Biosciences), Sca1-PE (Ly 6A/E, Caltag), c-Kit−APC (CD117, BD Biosciences) were used. For cell cycle analysis, the Hoechst 33342 dye was used according to the manufacturer's instructions (Molecular Probes). For BrdU incorporation, the APC-BrdU Flow Kit (BD Biosciences was used after a single intraperitoneal injection of BrdU (BD Biosciences, 1 mg per 6 g of body weight) and admixture of 1 mg/ml of BrdU (Sigma) to drinking water for 7 days. Surface staining for lineage markers was performed as above, Sca1-PE, c-Kit−APC/Cy5.5 (eBiosciences), and including CD34-FITC (BD Biosciences). For the apoptosis assay, DAPI dye and Annexin V (13D Biosciences) were used.

### CBC and PCR Analyses

p16^{INK4a} genotyping was done as described by Sharpless, et al (Sharpless, N. E. et al. Nature 413, 86-91 (2001)) and Y chromosome PCR as previously described (Cheng, T. et al. Science 287, 1804-1808 (2000)). Peripheral blood counts have been performed on Drew HemaVet 850.

### Gene Expression Analysis

RNA was isolated from sorted bone marrow populations using the PicoPure Kit (Arcturus Bioscience) according to the protocol. First-strand complementary DNA synthesis was synthesized using the High Capacity cDNA Archive Kit (Applied Biosystems) from 100 ng sample RNA, and amplification plots were generated using the Mx4000 Multiplex Quantitative QPCR System (Stratagene). To generate standard curves, cDNA from RB−/− cell line RNA (100 ng) was used as template in a five-fold dilution series. Sample cDNA was used undiluted. Relative expression was calculated using the delta Ct method. Pre-developed assays for Hprt-1, Bmi-1, Gfi-1 and hes-1 were purchased from Applied Biosystems with the following assay Ids, respectively: Mm00446968, Mm00776122, Mm00515853, and Mm00468601. Primers and Probes for p16^{INK4a} and ARF are as previously described. Krishnamurthy, J. et al. J. Clin. Invest. 114, 1299-1307 (2004))

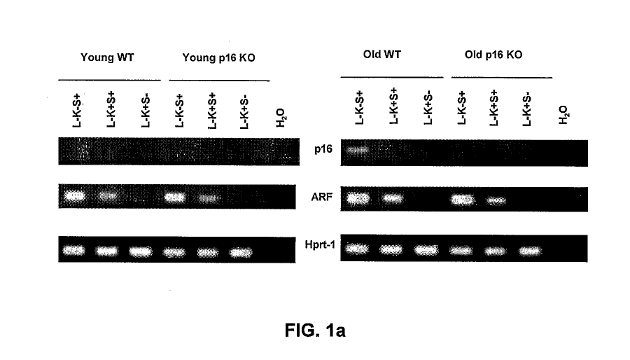

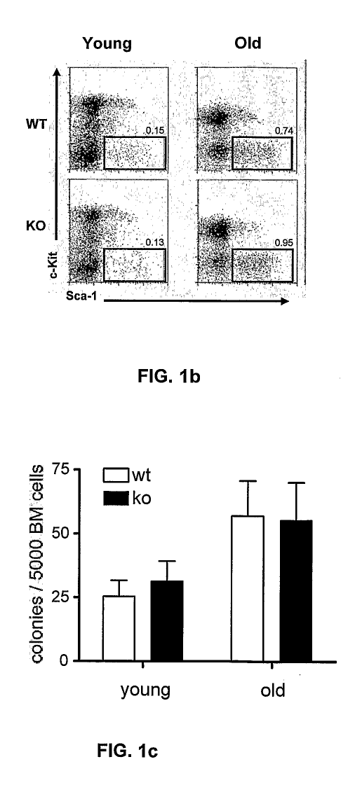

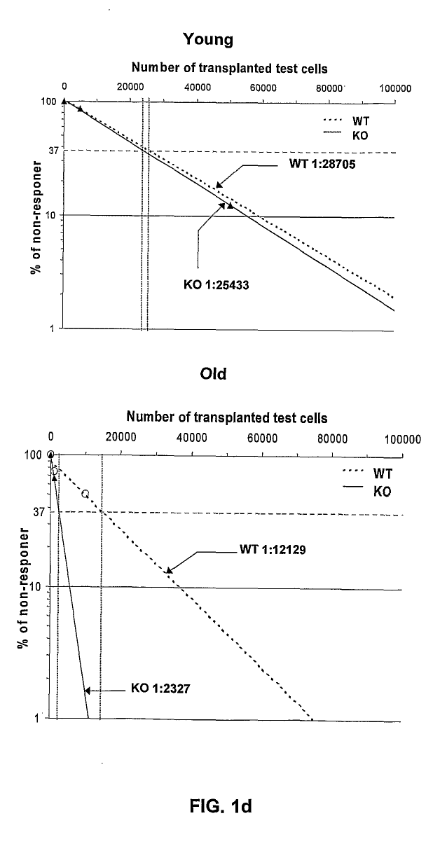

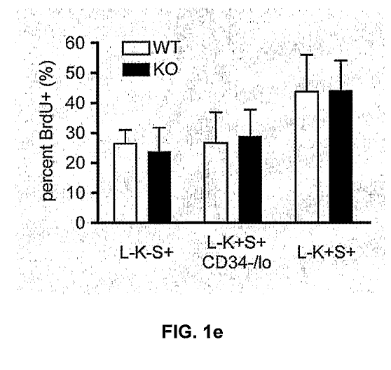

In young animals, p16^{INK4a} mRNA levels were below detection limits in whole bone marrow as well as in FACS-sorted populations enriched with primitive hematopoietic cells. However, in bone marrow of old animals, p16^{INK4a} mRNA became detectable in the Lin-negative/cKit-negative/Sca1-positive (LK−S+) population. This population has been identified to contain a more immature, deeply quiescent HSC than the LK+S+ population. (Doi, H. et al. Proc. Natl. Acad. Sci. U.S.A. 94, 2513-2517 (1997)) (Ortiz, M. et al. Immunity 10, 173-182 (1999)) In contrast to p16^{INK4a} expression, ARF was detectable in LK+S+ cells, although at higher levels in the LK−S+ population. In accord with previous findings in Lin− cells Krishnamurthy, J. et al. J. Clin. Invest. 114, 1299-1307 (2004), ARF mRNA also demonstrated an increase with aging, albeit more modestly than that observed for p16^{INK4a} (FIG. 1a). Hprt-1 expression was used as housekeeping control.

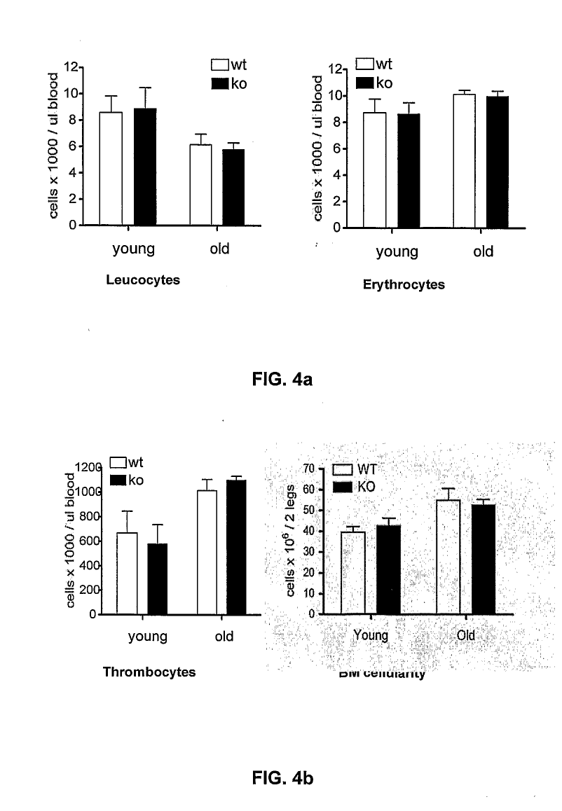

To assess the functional role of p16^{INK4a} in these compartments, mice selectively deficient for p16^{INK4a} with intact expression of ARF (Sharpless, N. E. et al. Nature 413, 86-91(2001)) were used. Confirming that p16^{INK4a} deficiency was not associated with a compensatory increase in ARF expression, nearly equivalent levels of ARF message were noted in primitive hematopoietic populations isolated from WT and p16^{INK4a-/-} BM (FIG. 1a). Bone marrow cellularity was assessed by enumerating the number of cells from both tibiae and femora of each animal. With advancing age, p16^{INK4a-/-} and WT mice exhibited comparable body size, peripheral blood counts and bone marrow cellularity (FIG. 4). Differential blood counts show no difference between the genotypes when age-matched animals were compared (young n=12, old n=5, p=n.s.) in any cell population (FIG. 4A). Bone marrow cellularity was assessed by enumerating the number of cells from both tibiae and femora of each animal (FIG. 4B). No differences in bone marrow cellularity were observed (young n=12, old n=4, p=n.s.).

Furthermore, no immunophenotypic differences were observed in bone marrow subpopulations (LK+S+, LK−S+ or LK+S−) derived from WT and p16^{INK4a-/-} mice at a young age (FIG. 1b). However, as mice from both genotypes age, a significant increase in the LK−S+ population was observed (FIG. 1b, n=9 for each genotype, p<0.01). It was in this population that p16^{INK4a} expression had been noted in aged wild type animals, indicating that an age-induced increase in p16^{INK4a} expression limits the number of LS+K− cells in vivo. Thus, immunophenotypic analysis of HSC-containing populations showed a significant increase of Lin−Sca1+c-kit− cells but not in Lin−Sca1-c-kit+ and Lin−Sca1+c-Kit+ over time in wild type, and p16^{INK4a} bone marrow were detectable(n=9; p(young/old) <0.01).

In an effort to determine whether the immunophenotypic subsets corresponded closely to functional subsets, the number of transient amplifying or progenitor cells present in mutant animals was enumerated by performing in vitro colony forming assays. Young p16^{INK4a-/-} mice showed a slight increase of colony forming cells (CFC) over their wild type counterparts. However, with increasing age, no differences in progenitor activity between the genotypes were detectable (FIG. 1c). Thus, with aging, the overall CFC-frequency increases, but p16^{INK4a} lose their progenitor advantage.

To determine whether mice lacking p16^{INK4a} have an altered number of functional HSCs within the bone marrow, competitive transplants were performed with limiting dilution analyses.

### Transplantation Assays

For serial transplantation, 3-4×10⁶ whole bone marrow cells from either 8 to 12 or 52 to 67 weeks old male FVB p16^{INK4a} WT and KO littermates were injected into lethally irradiated (10 Gy) 6 to 8 weeks old female recipient mice. CBC were obtained by tail vein nicking 4 weeks post transplantation. Six weeks post transplantation, recipients were used as donors for the next transplantation cycle and for in vitro assays. Transplants were discontinued when survival was below 50%.

Competitive Repopulation assay

For the competitive repopulation assay (CRA) with bone marrow cells from young mice, 5×10³, 5×10⁴, and 5×10⁵ WT or KO whole bone marrow cells were used from CD45.2 littermates (8 weeks old) mixed with 5×10⁵ CD45.1 (competitor) WT cells (8 weeks old). Recipients were 8-10 week-old CD45.1 B6.SJL female mice. For the competitive repopulation assay (CRA) with bone marrow cells from old mice, 1×10³, 1×10⁴⁴, and 1×10⁵ WT or KO whole bone marrow cells were used from CD45.2 littermates (52-60 weeks old) mixed with 2×10⁵ CD45.1 WT cells (12 weeks old). Recipients were 8-10 week-old CD45.1 B6.SJL female mice. Repopulation was assessed by flow cytometry at weeks 6 and 12 post transplant.

Peripheral blood was analyzed at 6 and 12 weeks post transplant to determine the degree of hematopoietic reconstitution and specific lineage contribution by the CD45.2-derived donor cells. When injected 1:1 with WT CD45.1-competing cells, p16^{INK4a-/-} donor cells from old mice gave rise to a significantly higher fraction of total peripheral blood than did their WT CD45.2 counterparts (p=0.00006), indicating a superior ability to compete and engraft in the absence of p16^{INK4a}. In contrast to marrow from old mice, no difference between WT and p16^{INK4a-/-} was noted when bone marrow was derived from young mice (p=0.9). The limiting dilution assay revealed a higher frequency of multi-lineage repopulating cells in p16^{INK4a}-deficient donor BM in old mice after 12 weeks of engraftment (p<0.04), while no difference in stem cell frequency between young WT and KO (12 weeks post transplant) was detectable (FIG. 1d). Thus, old (58 weeks C57B1/6) p16^{INK4a-/-} mice showed an increase number of long term repopulating hematopoietic stem cells compared with wild type control.

Since the total number of mononuclear cells per femur was unchanged between the genotypes, these data indicate an increase in the absolute number of long term repopulating cells in older animals null for p16^{INK4a}. The absence of p16^{INK4a} did not adversely affect differentiation capacity, as no difference was observed in the distribution of mature cells of different lineages between WT and KO donor cells. Therefore, there was an age-dependent effect of p16^{INK4a} on the number of hematopoietic stem cells. The presence of p16^{INK4a} restricts the hematopoietic stem cell pool in an aging organism.

Frequency and pool size of hematopoietic subpopulations can be affected by changes in cell cycle, apoptosis, or rate of transition to more mature compartments through differentiation. Since p16^{INK4a} is known to play an important role in cell cycle regulation in vitro, the impact of p16^{INK4a} deletion on the distribution of primitive hematopoietic cells was analyzed in various stages of the cell cycle. In flow cytometric analyses using Hoechst 33342, no differences in the frequency of cells in different cell cycle stages were detected in bone marrow populations from WT and p16^{INK4a-/-} mice.

As subtle differences in cell cycle activity might escape the “snap shot” detection by this method, efforts were made to enumerate the frequency of cycling cells over a longer period of time. Therefore, 5-bromodeoxyuridine (BrdU) was administered over a period of 7 days, and the percentage of BrdU+cells present within the primitive hematopoietic BM sub-populations was assessed (n=4, p=n.s.). No differences in the fraction of cells having initiated a division during the treatment period were detectable between young WT and p16^{INK4a-/-} animals (FIG. 1e). In fact, no effect of p16^{INK4a} on the rate of proliferation in primitive hematopoietic subpopulations was detectable in the presence or absence of p16^{INK4a} using BrdU incorporation. These data indicate that p16^{INK4a} expression does not affect HSC cell cycle kinetics in young animals, although it is not possible to rigorously exclude subtle effects on rare hematopoietic stem cells.

### Example 2

### p16^{INK4a} has No Effect on Cycling of Bone Marrow Stem Cells under Proliferative Stress of Sequential 5-Fluorouracil (5-FM) Treatment

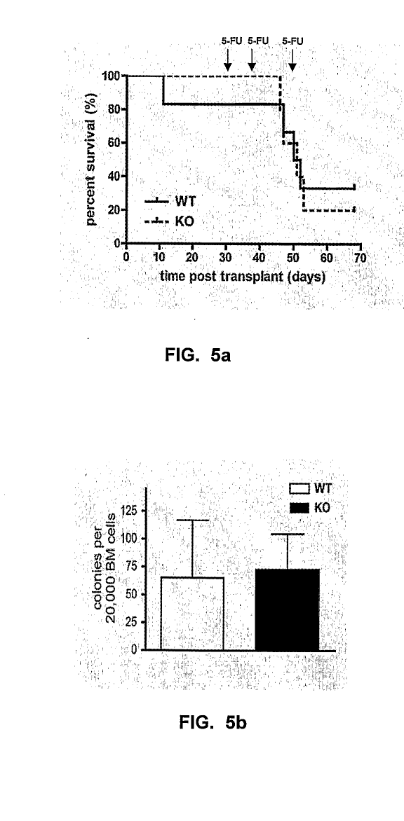

To confirm that the biological impact of p16^{INK4a} expression on aged bone marrow function might be uncovered by providing an exogenous stress to marrow homeostasis, 3×10⁶ WT or KO whole bone marrow cells were transplanted from young animals into lethally irradiated WT recipients, and the reconstituted recipients were exposed to repeated, weekly doses of 150 mg/kg 5-fluorouracil (5-FU), which specifically damages cycling cells. This protocol depletes cycling cells and provokes expansion and differentiation of the surviving, quiescent cells. Each round of treatment further stresses the population of non-cycling, primitive cells and, thus, audits the relative “depth” of the quiescent stem cell pool. Recipient mice were assayed for changes in survival or production of CFC, revealing no differences in either parameter (FIGS. 5A and 5B). Taken together, these data indicate that p16^{INK4a-/-} primitive hematopoietic cells or stem cells enter the cell cycle at a similar rate, as do their wild type littermates. However, an elevated proportion of the highly proliferative, more mature progenitor compartment appears to be cycling in the null mice. Despite the known role of p16^{INK4a} in cell cycle regulation in vitro, and despite the apparent increase in the stem cell pool in the p16^{INK4a-/-} animals, there does not appear to be altered stem cell cycling in the p16^{INK4a} deficient animals.

### Example 3

### p16^{INK4a} has No Effect on Frequency of Apoptotic Events in Primitive Hematopoietic Cells

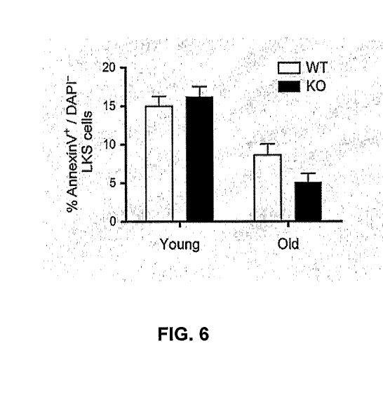

To determine whether the observed difference in stem cell number was instead due to changes in apoptotic rates, an Annexin V/DAPI assay was used. Freshly isolated bone marrow was stained for Lineage negative, Sca-1positive, c-Kit positive cells and co-stained with Annexin V and DAPI. No differences in the percentage of apoptotic cells (i.e., no effect from p16^{INK4a}) were detected between WT and KO in the LKS, LK−S+, or LK+S− populations in young, as well as in old, mice (FIG. 6). Taken together, these data indicate that the stem cell-enriched populations of bone marrow are disproportionately increased with age in the absence of p16^{INK4a}. Within the quantitative limits of the above assays, this finding cannot be attributed to discernable changes in cell cycling, apoptosis or differentiation capacity.

### Example 4

### p16^{INK4a} has an Age-Dependent Effect on Stem Cell Self-Renewal Potential

A signature function of stem cells is their ability to undergo self-renewing cell divisions, a feature critical for the sustained ability to maintain or repair tissues throughout life. Moreover, serial transplantation studies have shown that single clones of bone marrow cells are able to reconstitute lethally irradiated hosts in secondary, tertiary and quaternary transplants over a cumulative period that exceeds the lifespan of the donor. (Siminovitch, L. et al. J. Cell. Physiol., 23-31 (1964)) (Harrison, D. E. Nat. New Biol. 237, 220-222 (1972)) Thus, HSC have profound self-renewal capacity; however, cumulative evidence now demonstrates a measurable and inexorable decline in hematopoietic stem cell function including self-renewal, with advancing age. Ogden, D. A. et al. Transplantation 22, 287-293 (1976) (de Haan, G. et al. Blood 93, 3294-3301 (1999) Stem cell function affects longevity (Schlessinger, D. et al. Mech. Ageing Dev. 122, 1537-1553 (2001), and Van Zant, et al. demonstrated a mouse strain specific correlation of stem cell function with animal lifespan. (Van Zant, G., et al. J. Exp. Med. 171, 1547-1565 (1990)) Specifically, the HSC of short-lived DBA/2 mice exhibited a time dependent disadvantage when in competition with the HSC of long-lived C57B1/6 mice. (Van Zant, G., et al. J. Exp. Med. 171, 1547-1565 (1990))

In order to definitively address the question of whether p16^{INK4a} affects HSC self-renewal, serial bone marrow transplantation studies were performed with young (8-12 week old) or old (52-67 week old) donor mice. This assay is designed to examine the ability of a limited number of HSC clones to undertake a self-renewing rather than differentiation fate under physiologic pressure. 4-6×10⁶ bone marrow cells from FVB/n WT or p16^{INK4a-/-} mice were transplanted into lethally irradiated 6-8 week-old female FVB/n WT mice; after 6 weeks, recipients were euthanized, and 4-6×10⁶ of the harvested bone marrow cells were injected into new female irradiated recipients. This process was repeated an additional two times.

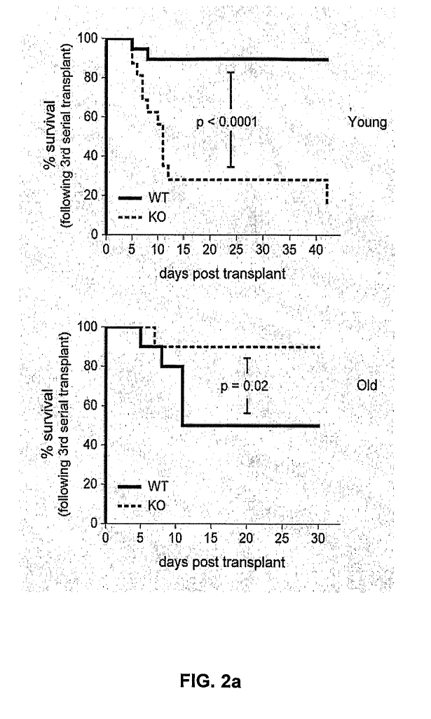

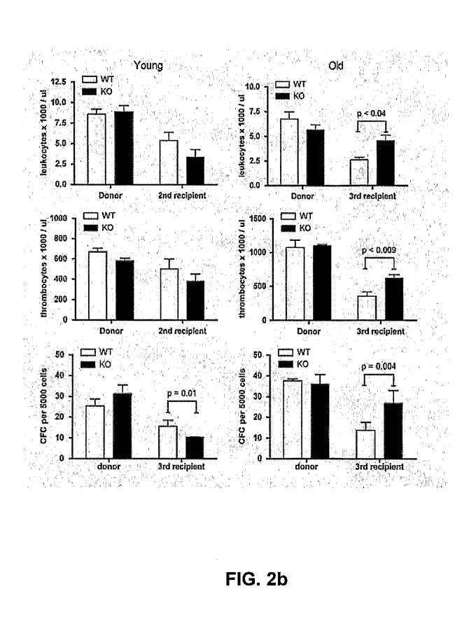

WT cells from older donors had reduced capacity to rescue transplanted recipients when compared with younger WT donors (note decreased survival after three serial transplants in FIG. 2a). Comparing young WT with young KO donors, an increase in mortality was observed among those receiving KO cells. The difference reached a significant level after the 3^{rd} transplantation round (p<0.0001) and peaked around day 10 post BMT (FIG. 2a). In effect, after the 3^{rd} transplant cycle, recipients of young p16^{INK4a} bone marrow showed a significant disadvantage in survival relative to their wild type counterpart. In contrast, recipients of old p16^{INK4a} bone marrow showed a significantly superior survival after the 3^{rd} transplant. In vitro assays were performed following each serial transplant to assay progenitor cell activity. A significant reduction in CFC frequency was detected from the p16^{INK4a-/-} BM recipients at 6 and 12 weeks following the 3^{rd} BMT, indicating that p16^{INK4a-/-} cells are unable to provide even short-term reconstitution following 3 rounds of in vivo expansion. These data indicate reduced self-renewal with subsequent stem cell exhaustion in HSCs from young mice lacking p16^{INK4a}.

In contrast, serial bone marrow transplantation using donor bone marrow from old mice demonstrated virtually reciprocal results. The KO recipients displayed significantly better survival (FIG. 2a, 3^{rd} cycle: n=20, p=0.02) and superior reconstitution, as measured by peripheral blood counts for all lineages (FIG. 2b). Consistent with these results, CFC frequency was higher in the KO recipients at the third transplantation (FIG. 2b). Recipients of 2^{nd} cycle of young p16^{INK4a-/-} bone marrow showed a tendency of decreased peripheral blood leukocytes and thrombocytes. Recipients of the 3^{rd} round of bone marrow from old mice showed the opposite results: P16^{INK4a-/-} recipients had more white blood cells and more thrombocytes.

Bone marrow cells of young p16^{INK4a-/-} recipients gave rise to less CFC-colonies than recipients of their wild type counterpart, while old bone marrow lacking p16^{INK4a} generated more CFC colonies after 3 rounds of transplantation. These observations indicate that p16^{INK4a} has a highly age-dependent effect on HSCs in very select functions. Specifically, sequential transplantation is altered. These data are considered a population-based measure of self-renewal, though it is recognized that other features of stem cell function may participate. Since no evidence of altered proliferation, differentiation, or apoptosis was detected under homeostatic conditions, the results likely reflect a higher frequency of self-renewing divisions in older p16^{INK4a} deficient stem cells.

The difference in sequential transplant capability of young versus old p16^{INK4a-/-} animals was striking. The effect in young animals was unexpected, since p16^{INK4a} expression was not found in young HSC under homeostatic conditions. However, when bone marrow from young mice after transplantation was examined, low level p16^{INK4a} expression was noted (data not shown), as has been seen by others under other conditions of stress. (Chkhotua, A. B. et al. Am. J. Kidney Dis. 41, 1303-1313 (2003)) (Chimenti, C. et al. Circ. Res. 93, 604-613 (2003)) The deleterious effect of p16^{INK4a} deficiency on HSC in this setting may be due to the known promoter competition between p16INK4a^{INK4a} and ARF, resulting in modest increases in ARF in p16^{INK4a} deletion. (Sharpless, et al. Oncogene 22, 5055-5059 (2003)) ARF expression has been shown to markedly increase HSC death. (Park I. K. et al. Nature 423, 302-305 (2003)) Conversely, the dual absence of p16^{INK4} and ARF or ARF alone has been shown to not result in any defect in serial transplantation in young animals. (Stepanova, L. et al. Blood (2005)) Indeed, the doubly deficient animal has a modest increase in self-renewal. (Stepanova, L. et al. Blood (2005)) It was hypothesized that the marked improvement in self-renewal with age in the absence of p16^{INK4a} was due to a mitigation of the molecular events induced by age-dependent increases in p16^{INK4a}.

### Example 5

### p16^{INK4a} has an Age-Dependent Effect on Expression of Self-Renewal Associated Genes in Primitive Subpopulations of Bone Marrow Cells

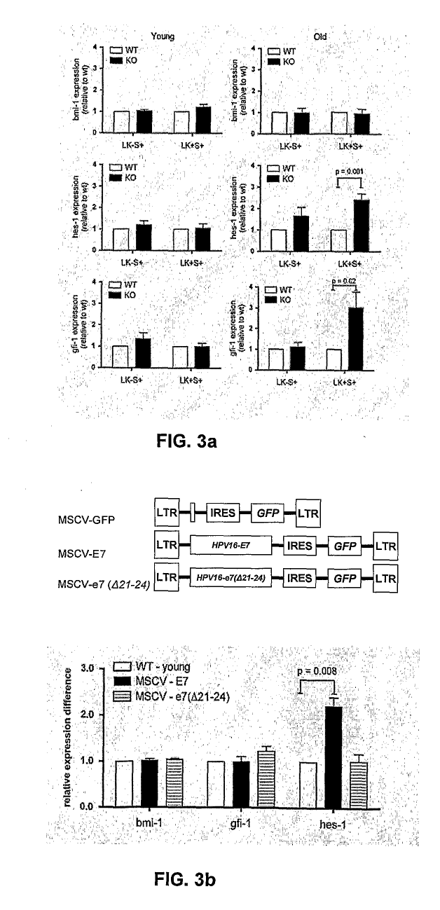

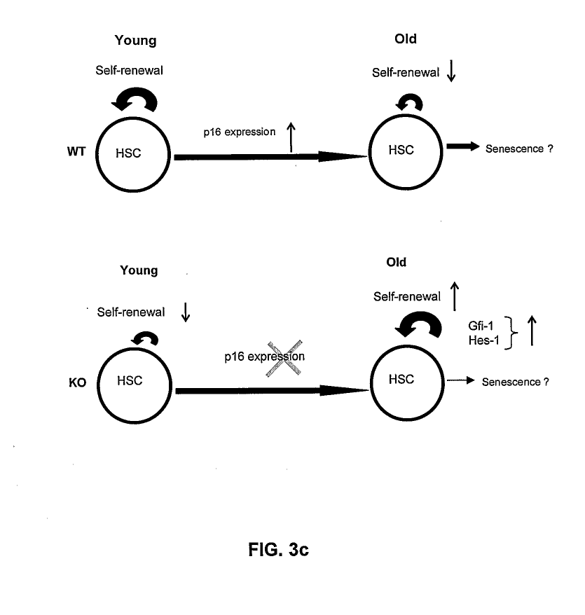

Age related-expression was first evaluated for select genes involved in HSC self-renewal. The polycomb gene bmi-1 is known to be essential for maintaining the hematopoietic stem cell pool. (Park, I. K. et al. Nature 423, 302-305 (2003)) Moreover, bmi-1 is known to suppress the expression of both genes of the Ink4a/Arf locus, p16^{INK4a} and ARF (Jacobs, J. J., et al. Nature 397, 164-168 (1999)). However, no differences in bmi-1 expression between WT and p16^{INK4a-/-} primitive cells in young and old mice were observed (FIG. 3a-b).

Hes-1 is known to be a downstream effector of notch-1 and has been established to play an important role in the self-renewal of hematopoietic stem cells (Kunisato, A. et al. Blood 101, 1777-1783 (2003)). Therefore, the expression of hes-1 was assayed within the primitive HSC compartments. In the LK+S+ and LK−S+subpopulations isolated from aged mouse bone marrow, a significant, approximately 2-fold, increase in hes-1 expression was found in p16^{INK4a-/-} LK+S+ compared to their WT counterparts (FIG. 3a-b). No differences in hes-1 expression were detected between young WT and KO mice, consistent with the observation that p16^{INK4a} expression is not detected in young cells under steady-state conditions.

The transcription factor gfi-1 has also been shown to regulate stem cell self-renewal (Hock, H. et al. Nature 431, 1002-1007 (2004)). Similar to the above-described findings with hes-1, no difference was detected in gfi-1 expression between WT and p16^{INK4a}-KO primitive hematopoietic cells in young animals. In contrast, old p16^{INK4a-/-} bone marrow LK+S+ cells showed an increase of gfi-1 expression compared to their WT littermates (FIG. 3a-b). In brief, real-time RT-PCR analyses were performed to assess the expression level of bmi-1, hes-1 and gfi-1 in FACS sorted Lin−c-Kit−Sca1+ and Lin−c-Kit+Sca1+ populations of young and old FVB/n mouse bone marrow. While no differences in expression of any of those genes between young p16^{INK4a+/+} and p16^{INK4a-/-} were detectable, hes-1 (n=3) and gfi-1 (n=3) was up-regulated in these populations of old p16^{INK4a} KO mice compared to their wild type littermates. Together, these data indicate that with increased age, p16^{INK4a} expression alters hes-1 and gfi-1 expression and p16^{INK4a} deficiency, hes-1 and gfi-1 levels both increase in stem cells in association with increased stem cell self-renewal.

Furthermore, the coding sequence of the human papillomavirus transforming protein HPV16-E7 was subcloned into the retroviral plasmid MSCV. An empty MSCV plasmid (MSCV-GFP) and a mutant variant of HPV-E7 with an inability to bind to Rb-protein MSCV-e7(Δ21-24) were used as controls. Sorted Lin−c-Kit+Sca1+ cells from old p16^{INK4a} FVB/n bone marrow were transduced with MSCV-virus containing HPV16-E7 construct or controls and cultured for 8 days prior RNA isolation and RT-PCR analysis. Expression of HPV-E7 caused a by-pass of the p16^{INK4a} effect on the Rb-pathway and showed a higher hes-1 expression compared to the control cells (n=3), while bmi-1 and gfi-1 expression remained unchanged. Consequently, bmi-1 transcription does not seem to play the key role in improving self-renewal in old mice lacking p16^{INK4a}, at least not in a steady state, non-transplanted setting.

Since p16^{INK4a} is known to act through binding to cdk4 and cdk6 and inhibiting Rb phosphorylation with consequent suppression of transcriptional activity of E2F, it was investigated whether the effect of p16^{INK4a} deficiency on gfi-1 or hes-1 transcript levels is mediated by an Rb-dependent effect. The transforming protein E7 of the human papilloma virus (HPV) binds to the Rb-family proteins derepressing E2F, resulting in transcriptional activation of downstream proteins. The coding sequence of the HPV-E7-protein was cloned into an MSCV plasmid and over-expressed in a stable transduction of old p16^{INK4a +/+} LK+S+ cells. A similar experiment with LK−S+ cells was not possible, as these cells did not grow in vitro, as also noted by others (Doi, H. et al. Proc. Natl. Acad. Sci. U.S.A. 94, 2513-2517 (1997)) (Ortiz, M. et al. Immunity 10, 173-182 (1999)). As controls, an empty MSCV-vector and a mutant E7 without the ability to bind Rb (E7 Δ21-24 (Phelps, W. C., et al. J. Virol. 66, 2418-2427 (1992))) were used.

Two days following transduction, LK+S+ cells were sorted for GFP+ cells and cultured for additional 8 days prior to RNA isolation and gene expression analysis. This additional cell culture time was enabled the up regulation of p16^{INK4a} expression in LK+S+ cells. In three independent experiments, cells transduced with the MSCV-E7 construct exhibited a 2-fold increase in hes-1 expression compared to the MSCV-empty vector control. However, no differences in gfi-1 or bmi-1 expression between MSCV-E7 and the vector controls were detected, suggesting that the elevation of gfi-1 observed ex vivo in aged p16^{INK4a}-KO cells may be due to a Rb-independent or indirect, more downstream pathway or gfi-1 may be a cell non-autonomous target of p16^{INK4a} (FIG. 3c).

Taken together, these data indicate an age-dependent effect for p16^{INK4a} on the self-renewal of hematopoietic stem cells. These data demonstrate the link of a stem cell aging phenotype specifically with p16^{INK4a}. Since stem cells provide the basis for tissue maintenance over time, p16^{INK4a} may then be considered a molecular focal point for some of the manifestations of age on tissue function. Altering p16^{INK4a} boosted stem cell self-renewal in old mice and enhanced animal endurance of the physiologic stress of transplantation. The effect of p16^{INK4a} on stem cell self-renewal observed herein was not related to a change in proliferation kinetics, but, rather, to a change in proliferation outcome, self-renewal. Therefore, it is likely due to p16^{INK4a} E2F and non-E2F mediated transcription events rather than direct interaction with specific cycling components. p16^{INK4a} modifies stem cell aging by altering the capacity of stem cells to self-renew in association with age-dependent alteration of self-renewal gene expression. Thus, modulating p16^{INK4a} can serve as a means of attenuating age-related phenotypes on the stem cell level.

Although the foregoing invention has been described in some detail by way of illustration and example for purposes of clarity and understanding, it will be apparent to those skilled in the art that certain changes and modifications can be practiced. Therefore, the description and examples should not be construed as limiting the scope of the invention, which is delineated by the appended numbered claims.

1. A method of promoting self-renewal of a stem cell that expresses p16^{INK4a}, the method comprising the step of:
contacting the stem cell with an effective amount of an inhibitor of p16^{INK4a}, thereby promoting self-renewal of the stem cell.

2. The method of claim 1, wherein the inhibitor of p16^{INK4a} reduces the expression of p16^{INK4a}.

3. The method of claim 2, wherein the inhibitor of p16^{INK4a} is selected from the group consisting of a compound that can destabilize or reduce the levels of p16^{INK4a} mRNA, a compound that can reduce translation of p16^{INK4a} mRNA, a compound that can hypermethylate p16^{INK4a}, telomerase reverse transcriptase (hTERT), inhibitor of DNA binding/differentiation (Id, or Id-1), latent membrane protein (LMP1), helix-loop-helix transcription factor TAL1/SCL, dioxin, and cyclo-oxygenase 2 (COX-2).

4. The method of claim 1, wherein the inhibitor of p16^{INK4a} reduces the activity of p16^{INK4a}.

5. The method of claim 4, wherein the inhibitor of p16^{INK4a} is selected from the group consisting of a p16^{INK4a} antibody, a compound that can hypermethylate p16^{INK4a}, telomerase reverse transcriptase (hTERT), cutaneous human papillomavirus type 16 (HPV1 6) E7 protein, and cyclin D1.

6. The method of claim 1, wherein the stem cell is a bone marrow derived stem cell.

7. The method of claim 1, wherein the stem cell is a hematopoietic stem cell.

8. The method of claim 1, wherein the stem cell is selected from the group consisting of a mesenchymal, skin, neural, intestinal, liver, cardiac, prostate, mammary, kidney, pancreatic, retinal and lung stem cell.

9. The method of claim 1, wherein the stem cell is contacted ex vivo.

10. The method of claim 1, wherein the stem cell is contacted in vivo.

11. The method of claim 1, wherein the expression of hes-1 and gfi-1 are increased in the stem cell.

12. A packaged pharmaceutical comprising an inhibitor of p16^{INK4a} and instructions for using said inhibitor to promote self-renewal of a stem cell that expresses p16^{INK4a} in accordance with the method of claim 1.

13. A method of increasing the amount of self-renewing stem cells in a non-infant subject in need thereof, the method comprising the steps of:
contacting an isolated population of cells comprising stem cells with an effective amount of an inhibitor of p16^{INK4a} ex-vivo; and
administering the cells to the non-infant subject, thereby increasing the amount of self-renewing stem cells in the non-infant subject.

14. The method of claim 13, wherein the inhibitor of p16^{INK4a} reduces the expression of p16^{INK4a}.

15. The method of claim 14, wherein the inhibitor of p16^{INK4a} is selected from the group consisting of a compound that can destabilize or reduce the levels of p16^{INK4a} mRNA, a compound that can reduce translation of p16^{INK4a} mRNA, a compound that can hypermethylate p16^{INK4a}, telomerase reverse transcriptase (hTERT), inhibitor of DNA binding/differentiation (Id, or Id-1), latent membrane protein (LMP1), helix-loop-helix transcription factor TAL1/SCL, dioxin, and cyclo-oxygenase 2 (COX-2).

16. The method of claim 13, wherein the inhibitor of p16^{INK4a} reduces the activity of p16^{INK4a}.

17. The method of claim 16, wherein the inhibitor of p16^{INK4a} is selected from the group consisting of a p16^{INK4a} antibody, a compound that can hypermethylate p16^{INK4a}, telomerase reverse transcriptase (hTERT), cutaneous human papillomavirus type 16 (HPV16) E7 protein, and cyclin D1.

18. The method of claim 13, wherein the population of cells is obtained from the non-infant subject.

19. The method of claim 13, wherein the population of cells comprise bone marrow cells.

20. The method of claim 13, wherein the population of cells is Lin⁻, cKit⁻ and Sca1⁺.

21. The method of claim 13, wherein the stem cells comprise hematopoietic stem cells.

22. The method of claim 13, wherein the expression of hes-1 and gfi-1 are increased in the stem cells.

23. The method of claim 13, wherein the non-infant subject is a human.

24. The method of claim 13, wherein the non-infant subject is at least 18 years old.

25. The method of claim 13, wherein the cells are administered to the non-infant subject during a bone marrow transplant.

26. A packaged pharmaceutical comprising an inhibitor of p16^{INK4a} and instructions for using said inhibitor to increase the amount of self-renewing stem cells in a non-infant subject in need thereof in accordance with the method fo claim 13.

27. A method of maintaining self-renewal of a stem cell that does not express p16^{INK4a}, the method comprising:
contacting the stem cell with an inhibitor of p16^{INK4a}, thereby maintaining self-renewal of the stem cell.

28. The method of claim 27, wherein the inhibitor of p16^{INK4a} reduces the expression of p16^{INK4a}.

29. The method of claim 28, wherein the inhibitor of p16^{INK4a} is selected from the group consisting of a compound that can destabilize or reduce the levels of p16^{INK4a} mRNA, a compound that can reduce translation of p16^{INK4a} mRNA, a compound that can hypermethylate p16^{INK4a}, telomerase reverse transcriptase (hTERT), inhibitor of DNA binding/differentiation (Id, or Id-1), latent membrane protein (LMP1), helix-loop-helix transcription factor TAL1/SCL, dioxin, and cyclo-oxygenase 2 (COX-2).

30. The method of claim 27, wherein the inhibitor of p16^{INK4a} reduces the activity of p16^{INK4a}.

31. The method of claim 30, wherein the inhibitor of p16^{INK4a} is selected from the group consisting of a p16^{INK4a} antibody, a compound that can hypermethylate p16^{INK4a}, telomerase reverse transcriptase (hTERT), cutaneous human papillomavirus type 16 (HPV16) E7 protein, and cyclin D1.

32. The method of claim 27, wherein the stem cell is a bone marrow derived stem cell.

33. The method of claim 27, wherein the stem cell is a hematopoietic stem cell.

34. The method of claim 27, wherein the stem cell is selected from the group consisting of a mesenchymal, skin, neural, intestinal, liver, cardiac, prostate, mammary, kidney, pancreatic, retinal and lung stem cell.

35. The method of claim 27, wherein the stem cell is contacted ex vivo.

36. The method of claim 35, wherein the stem cell is provided to a subject in a bone marrow transplant after it is contacted ex vivo.

37. The method of claim 27, wherein the stem cell is contacted in vivo.

38. The method of claim 27, wherein the expression of hes-1 and gfi-1 are increased in the stem cell.

39. A packaged pharmaceutical comprising A packaged pharmaceutical comprising an inhibitor of p16^{INK4a} and instructions for using said inhibitor to maintain self-renewal of a stem cell that does not express p16^{INK4a} in accordance with the method of claim 27.

40. A method for enhancing engraftment of a stem cell that expresses p16^{INK4a} into a tissue of a subject, the method comprising
contacting the stem cell with an effective amount of an inhibitor of p16^{INK4a} ex vivo; and
providing the stem cell to the subject, thereby enhancing engraftment of the stem cell into a tissue of a subject.

41. The method of claim 40, wherein the inhibitor of p16^{INK4a} reduces the expression of p16^{INK4a}.

42. The method of claim 41, wherein the inhibitor of p16^{INK4a} is selected from the group consisting of a compound that can destabilize or reduce the levels of p16^{INK4a} mRNA, a compound that can reduce translation of p16^{INK4a} mRNA, a compound that can hypermethylate p16^{INK4a}, telomerase reverse transcriptase (hTERT), inhibitor of DNA binding/differentiation (Id, or Id-1), latent membrane protein (LMP1), helix-loop-helix transcription factor TAL1/SCL, dioxin, and cyclo-oxygenase 2 (COX-2).

43. The method of claim 40, wherein the inhibitor of p16^{INK4a} reduces the activity of p16^{INK4a}.

44. The method of claim 43, wherein the inhibitor of p16^{INK4a} is selected from the group consisting of a p16^{INK4a} antibody, a compound that can hypermethylate p16^{INK4a}, telomerase reverse transcriptase (hTERT), cutaneous human papillomavirus type 16 (HPV16) E7 protein, and cyclin D1.

45. The method of claim 40, wherein the stem cell is a bone marrow derived stem cell.

46. The method of claim 40, wherein the stem cell is a hematopoietic stem cell.

47. The method of claim 40, wherein the stem cell is selected from the group consisting of a mesenchymal, skin, neural, intestinal, liver, cardiac, prostate, mammary, kidney, pancreatic, retinal and lung stem cell.

48. The method of claim 40, wherein the expression of hes-1 and gfi-1 are increased in the stem cell.

49. The method of claim 40, wherein the tissue comprises bone marrow.

50. The method of any one of claims 1, 13, 27, and 40, further comprising the step of obtaining the inhibitor of p16^{INK4a}.

51. A packaged pharmaceutical comprising an inhibitor of p16^{INK4a} and instructions for using said inhibitor to enhance engraftment of a stem cell that expresses p16^{INK4a} into a tissue of a subject in accordance with the method of claim 40.

52. A method of identifying an inhibitor of p16^{INK4a}, wherein the inhibitor promotes the self-renewal of stem cells, the method comprising:
contacting an isolated population of cells comprising stem cells that express p16^{INK4a} with an agent suspected of being an inhibitor of p16^{INK4a}; and
detecting an increase in the total number of long term repopulating cells, thereby identifying an inhibitor of p16^{INK4a} that promotes the self-renewal of the stem cells.

53. The method of claim 52, wherein the inhibitor of p16^{INK4a} reduces the expression of p16^{INK4a}.

54. The method of claim 52, wherein the inhibitor of p16^{INK4a} reduces the activity of p16^{INK4a}.

55. The method of claim 52, wherein the population of cells is obtained from a non-infant subject.

56. The method of claim 52, wherein the population of cells comprise bone marrow cells.

57. The method of claim 52, wherein the population of cells is Lin⁻, cKit− and Sca1⁺.

58. The method of claim 52, wherein the stem cells comprise hematopoietic stem cells.

59. The method of claim 52, wherein the expression of hes-1 and gfi-1 are increased in the stem cells.

60. The method of claim 52, further comprising the step of obtaining the agent.

61. A kit for promoting self-renewal of a stem cell that expresses p16^{INK4a} comprising an inhibitor of p16^{INK4a}, and instructions for using the inhibitor of p16^{INK4a} to promote self-renewal of a stem cell that expresses p16^{INK4a} in accordance with the method of claim 1.

62. A kit for increasing the amount of self-renewing stem cells in a non-infant subject in need thereof comprising an inhibitor of p16^{INK4a}, and instructions for using the inhibitor of p16^{INK4a} to increase the amount of self-renewing stem cells in a non-infant subject in need thereof in accordance with the method of claim 13.

63. A kit for maintaining self-renewal of a stem cell that does not express p16^{INK4a} comprising an inhibitor of p16^{INK4a}, and instructions for using the inhibitor of p16^{INK4a} to maintain self-renewal of a stem cell that does not express p16^{INK4a} in accordance with the method of claim 27.

64. A kit for enhancing engraftment of a stem cell that expresses p16^{INK4a} into a tissue of a subject comprising an inhibitor of p16^{INK4a}, and instructions for using the inhibitor of p16^{INK4a} to enhance engraftment of a stem cell that expresses p16^{INK4a} into a tissue of a subject in accordance with the method of claim 40.

65. The method of claim 13, wherein the subject has a disorder selected from the group consisting of: thrombocytopenia, anemia, lymphocytopenia, lymphorrhea, lymphostasis, erythrocytopenia, erythrodegenerative disorder, erythroblastopenia, leukoerythroblastosis; erythroclasis, thalassemia, myelofibrosis, thrombocytopenia, disseminated intravascular coagulation (DIC), immune thrombocytopenic purpura (ITP), HIV inducted ITP, myelodysplasia, thrombocytotic disease, thrombocytosis, neutropaenia, myelo-dysplastic syndrome, infection, mmunodeficiency, rheumatoid arthritis, lupus, immunosuppression, systemic lupus erythematosus, rheumatoid arthritis, auto-immune thyroiditis, scleroderma, and inflammatory bowel disease.
